(19) United States
(12) Patent Application Publication
(10) Pub. No.: US 2016/0114129 A1
(43) Pub. Date: Apr. 28, 2016

(54) ACTUATING CATHETER LUER AND HUB DEVICES

(71) Applicant: AngioDynamics, Inc., Latham, NY (US)

(72) Inventor: Carol L. Lancette, Fort Ann, NY (US)

(73) Assignee: AngioDynamics, Inc., Latham, NY (US)

(21) Appl. No.: 14/663,530

(22) Filed: Mar. 20, 2015

Related U.S. Application Data
(63) Continuation-in-part of application No. 14/523,025, filed on Oct. 24, 2014.

(51) Int. Cl. A61M 25/00; A61M 39/06; A61M 39/24

(52) U.S. Cl. CPC A61M 25/0075; A61M 25/0069; A61M 25/0097; A61M 39/24; A61M 39/06; A61M 25/0026; A61M 2025/0079; A61M 2025/0078; A61M 2025/0076; A61M 2039/1044

(57) ABSTRACT

A hub for a single lumen or multi-lumen catheter is disclosed. In certain embodiments, the hub features integrated luers, and does not require the use of extension tubes or extension tube clamps. The hub has an actuating mechanism for closing fluid access to catheter lumens. In certain embodiments, the hub has a proximal and distal housing, and the proximal housing can be rotated, pulled or pushed relative to the distal housing for actuating the mechanism. In certain embodiments, the hub includes a valve, such as an iris valve, for opening and closing fluid access to lumens, or for regulating fluid flow rates through lumens. In certain embodiments, a switching element is used to open and close fluid access to hub lumens.

### CROSS-REFERENCE TO RELATED APPLICATIONS

This application is a continuation-in-part of U.S. patent application Ser. No. 14/523,025 filed on Oct. 24, 2014, the entire disclosure of which is incorporated by reference herein in its entirety.

### BACKGROUND OF THE INVENTION

Medical devices such as catheters are commonly used by medical professionals for establishing fluid access with a target area within the human body. Common types of catheters include dialysis catheters, peripherally inserted central catheters, midline catheters, central venous catheters and drainage catheters. The type of catheter used on a particular patient is typically based on a number of factors, including the procedure being performed and the area within the human body that needs to be accessed. One typical use is for the infusion and/or aspiration of fluid to and from a vessel of a human body. One common feature of these types of conventional catheters is that they often have a proximal end configuration that includes a hub, and extension tube, a luer, and a clamp surrounding the extension tube for closing-off fluid access to the catheter lumen between procedures. Closing fluid access to the catheter lumen while the catheter is not in use is an important safety precaution for minimizing risk of air embolism, exsanguination and contamination.

A conventional dialysis catheter is shown in FIG. 31. The main body of the catheter 300 features an elongate shaft 312 having a proximal end 314 and a distal end 316. The elongate shaft 312 is typically a dual lumen shaft that terminates at the distal end 316 in a step tip (as shown) or split tip configuration. The hub 318 provides a transition and bifurcation point for each of the catheter lumens to split off individually into separated tubing at the proximal end 314 of the device. In addition, the hub 318 often doubles as an anchor point for the catheter, often including one or more attachment elements such as a suture wing for anchoring the device to the body of the patient. Each separated lumen branches out into a respective extension tube 324, 326. The arterial lumen branches out to a first extension tube 324, which is surrounded by a clamp 328. The proximal end of the arterial lumen extension tube 324 terminates in a luer 320. Likewise, the venous lumen branches out to a second extension tube 326, which is surrounded by a clamp 330. The proximal end of the venous lumen extension tube 326 also terminates in a luer 322. The clamps 328, 330 can be closed to seal off the extension tube 324, 326 lumens.

Problems may arise with the use of extension tubes and clamps to seal off fluid access. For instance, frequent opening and closing of the clamps can start to weaken the extension tube, which may ultimately require an extension tube repair, or replacement of the entire catheter. Further, clamps surrounding the extension tube are limited to clamping the extension tube, and do not typically extend to more proximal points on the device, such as the luer. So even when the clamp is closed, the parts of the extension tube lumen and luer interior that are proximal of the clamp remain exposed to fluid and air communication, giving rise to potential contamination. It would be desirable to reduce the proximal end weight and bulk of the catheter, while reducing production costs associated with manufacturing the catheter. Still further, it would be desirable to fluidly seal lumens on the device at a point more proximal than conventional extension tube and clamp configurations. In addition, it would be desirable to close off proximal end fluid access to lumens, without requiring a separate accessory component such as an end cap or other accessory device. Further, it would be beneficial to open and close fluid access to the catheter while minimizing wear to an extension tube, which will lower the incidence of extension tubing replacement procedures and/or catheter exchanges.

### SUMMARY OF THE INVENTION

In one aspect, a hub for a catheter is described. The hub may include a proximal housing having a first proximal lumen in fluid communication with a first luer, and a distal housing having a first distal lumen. The proximal housing is attached to the distal housing and configured to rotate relative to the distal housing between an open state and a closed state, the open state establishes fluid communication between the first proximal lumen and the first distal lumen, and the closed state blocks fluid communication between the first proximal lumen and the first distal lumen.

In another aspect, a vascular access catheter is described. The vascular access catheter may include an elongate flexible shaft connected to and in fluid communication with a hub, the hub including a proximal housing having a first proximal lumen in fluid communication with a first luer, and a distal housing having a first distal lumen, where the proximal housing is attached to the distal housing and configured to rotate relative to the distal housing between an open state and a closed state, where the open state establishes fluid communication between the first proximal lumen and the first distal lumen, and where the closed state blocks fluid communication between the first proximal lumen and the first distal lumen.

In yet another aspect, a hub for controlling flow of a fluid along a flow path is described. The hub may include a proximal housing having a first proximal lumen in fluid communication with a first luer, and a distal housing having a first distal lumen and a first stem, where the proximal housing is attached to the distal housing and configured to slide relative to the distal housing between an open state and a closed state, where the open state establishes fluid communication between the first proximal lumen and the first distal lumen, and where the closed state blocks fluid communication between the first proximal lumen and the first distal lumen by interfacing the first stem with the proximal lumen.

In yet another aspect, a hub for controlling flow of a fluid along a flow path is described. The hub may include a proximal housing portion having a first proximal lumen in fluid communication with a first luer, a distal housing portion having a first distal lumen, and a switching element having a first switching lumen, where the switching element is configured to rotate between an open state and a closed state, and is disposed between the proximal housing portion and the distal housing portion, where the open state establishes fluid communication between the first proximal lumen and the first distal lumen by aligning the first proximal lumen and the first distal lumen with the first switching lumen, and where the closed state blocks fluid communication between the first proximal lumen and the first distal lumen by aligning the first proximal lumen and the first distal lumen with a solid portion of the switching element.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a side view of a dialysis catheter according to an exemplary embodiment of the invention;

FIG. 2 is a side view of a luer in closed position according to an exemplary embodiment of the invention;

FIG. 3 is a side view of the luer shown in FIG. 2 in open position;

FIG. 4 is a cross-sectional side view of the luer shown in FIG. 2 in closed position;

FIG. 5 is a cross-sectional side view of the luer shown in FIG. 2 in open position;

FIG. 6 is a cross-sectional view of the luer shown in FIG. 2 along cross section 6-6 shown in FIG. 5;

FIG. 7 is a cross-sectional side view of a luer with an offset stem in open position according to an exemplary embodiment of the invention;

FIG. 8 is a cross-sectional view of the luer shown in FIG. 7 along cross section 8-8;

FIG. 9 is a side view of a luer with a guide wire opening in a lower housing and in closed position according to an exemplary embodiment of the invention;

FIG. 10 is a cross-sectional side view of the luer shown in FIG. 9;

FIG. 11 is a side view of a luer with a twisting mechanism according to an exemplary embodiment of the invention;

FIG. 12 is a cutaway view of the valve mechanism inside of the luer shown in FIG. 11 in open position;

FIG. 13 is a cutaway view of the valve mechanism inside of the luer shown in FIG. 11 in closed position;

FIG. 14 is a side view of a rotating hub mechanism according to an exemplary embodiment of the invention.

FIG. 15 is a side view of the rotating hub mechanism shown in FIG. 14, with the proximal housing of the hub twisted clockwise 90 degrees.

FIG. 16 is a cross-sectional view of the rotating hub mechanism shown in FIG. 14, taken across cross section A-A′.

FIG. 17 is a cross-sectional view of the rotating hub mechanism and configuration shown in FIG. 15, taken across cross section B-B′.

FIG. 18 is a side view of a hub in an open position, according to an exemplary embodiment of the invention.

FIG. 19 is a side view of the hub shown in FIG. 18 in a closed position.

FIG. 20 is a partial cross-sectional side view of the hub shown in FIG. 18 in an open position.

FIG. 21 is a partial cross-sectional side view of the hub shown in FIG. 19 in a closed position.

FIG. 22 is a side view of hub including an iris valve for a single lumen catheter according to an exemplary embodiment of the invention.

FIG. 23 is a side view of a hub configuration including an iris valve for a dual lumen catheter according to an exemplary embodiment of the invention.

FIGS. 24A-24C are top views of an iris valve according to an aspect of exemplary embodiments of the invention. FIG. 24A shows the iris valve in a closed position, FIG. 24B shows the iris valve in a partially open position, and FIG. 24C shows the iris valve in an open position.

FIG. 25 is a side view of a hub with a switching mechanism set to an open position according to an exemplary embodiment of the invention.

FIG. 26 is a side view of the hub with a switching mechanism shown in FIG. 25, with the switching mechanism set to a closed position.

FIG. 27 is a cross-sectional view of the switching element taken across cross section C-C′ while set to the open position.

FIG. 28 is a cross-sectional view of the switching element taken across cross section D-D′ while set to the closed position.

FIG. 29 is a cross-sectional view of a switching element set to an open position, having both lumens open, according to an alternate embodiment.

FIG. 30 is a cross-sectional view of the switching element shown in FIG. 29, with only a second lumen open.

FIG. 31 is a cross-sectional view of the switching element shown in FIG. 29, set to a closed position, having both lumens closed.

FIG. 32 is a cross-sectional view of the switching element shown in FIG. 29, with only a first lumen open.

FIG. 33 is a side view of a hub having a twisting mechanism according to an exemplary embodiment of the invention.

FIG. 34 is a side view of the hub of FIG. 29, with the proximal housing twisted counterclockwise 90 degrees.

FIG. 35 is a side view of a prior art dialysis catheter.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein are improved catheter luer and hub devices, and methods of using the same.

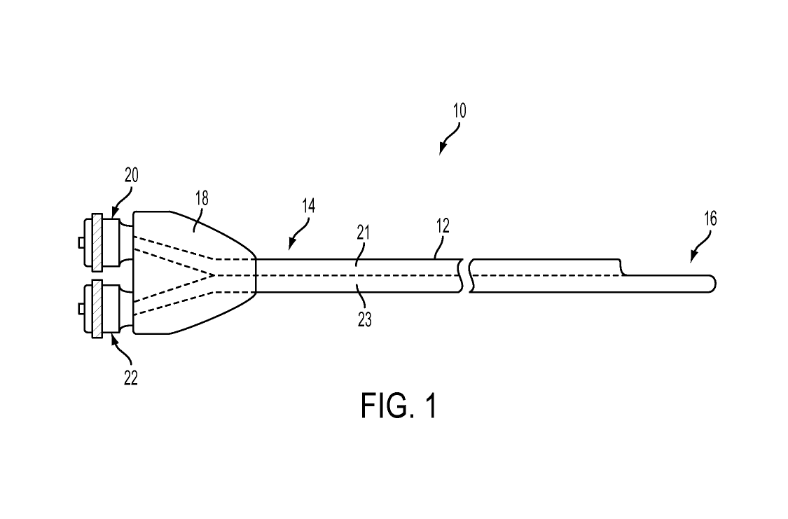

A first exemplary embodiment of the invention is shown in the dialysis catheter of FIG. 1. The dialysis catheter 10 has a flexible elongate shaft 12 that extends from a proximal end 14 to a distal end 16. The elongate shaft 12 has an arterial lumen 21 for removing blood from the body and a venous lumen 23 for returning blood to the body during a dialysis treatment. The distal end 16 of the shaft 12 terminates in a step tip configuration, offsetting the distal openings of each lumen 21, 23. The step tip configuration is known in a common configuration for minimizing blood recirculation rates during treatment. The proximal end of the lumens 21, 23 continue through the hub 18, where they bifurcate and extend into fluid communication with a respective luer 20, 22. Each luer can be coded with a label and/or color to indicate whether it is the luer for the arterial lumen or the venous lumen. The luers 20, 22 are integrally formed with the catheter hub 18.

The shaft 12 can be made of a medical grade polymer, such as a medical grade urethane. The luers 20, 22 can be integrally formed with or attached to the hub 18 by a number of methods known in the art. For instance, the hub 18 and luers 20, 22 can be attached utilizing a chemical bond, such as cyclohexanone, or an adhesive bond, such as a UV curable adhesive. Injection molding and/or overmolding techniques can also be used to mold the hub 18 and luers 20, 22 as integral components. It will be understood by those having ordinary skill in the art that if desired, the luers described herein can also be utilized with extension tubes and will maintain certain advantages described herein for a clampless catheter. Chemical bonds, adhesive bonds and overmolding techniques described above can be utilized along with techniques known in the art for attaching the luers described herein to extension tubes.

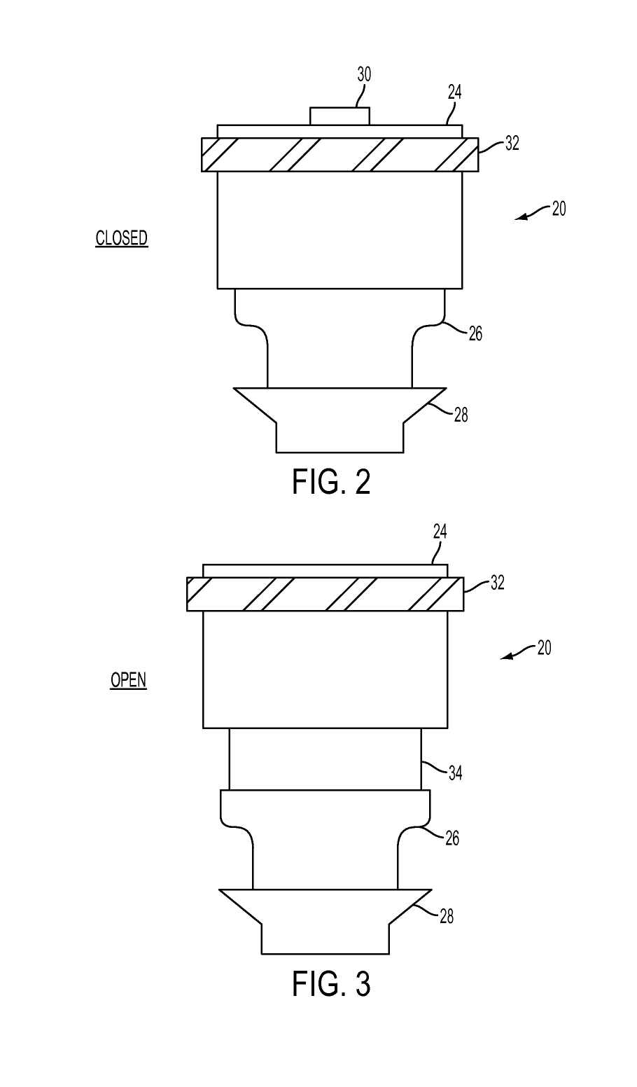

With reference to FIGS. 2 and 3, a luer 20 is shown in closed and open positions respectively according to an exemplary embodiment of the invention. The luer 20 includes a proximal housing 24 and a distal housing 26 that are slidably connected to each other in a mating configuration. The distal housing may include an element such as a barb 28 for facilitating attachment to a hub or an extension tube. The proximal housing 24 has a threaded element 32 so that the device can be attached to tubing connections such as tubing connections for a dialysis machine or syringes for flushing the device. The slidable connection between the proximal housing 24 and the distal housing 26 allows medical practitioners to easily slide or “pop” the luer 20 from a closed configuration (shown in FIG. 2) to an open configuration (shown in FIG. 3), using a simple grasping, pulling or pushing motion with one hand.

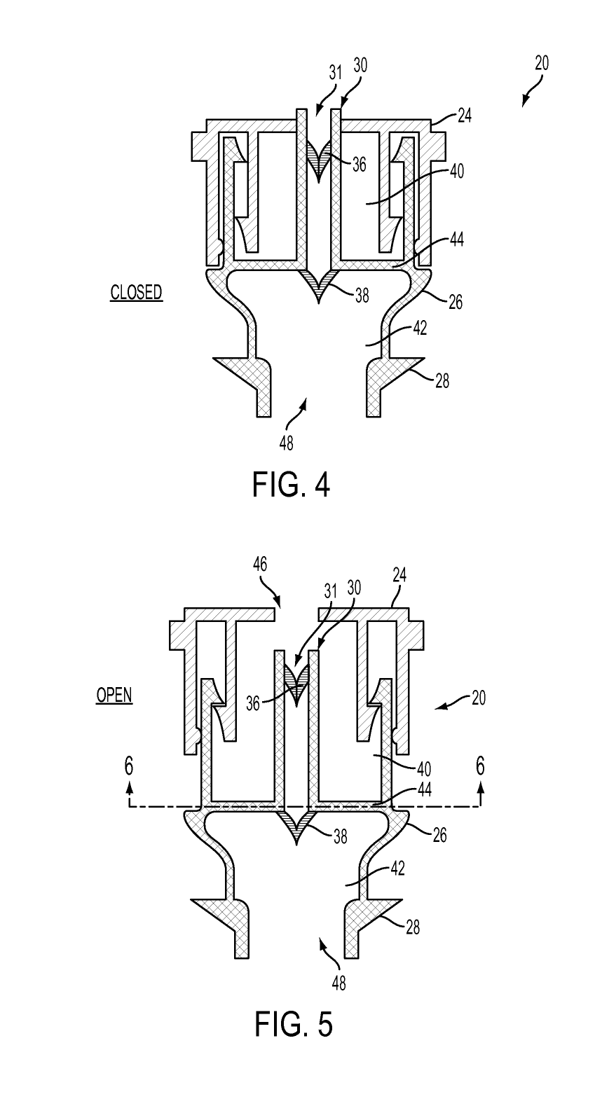

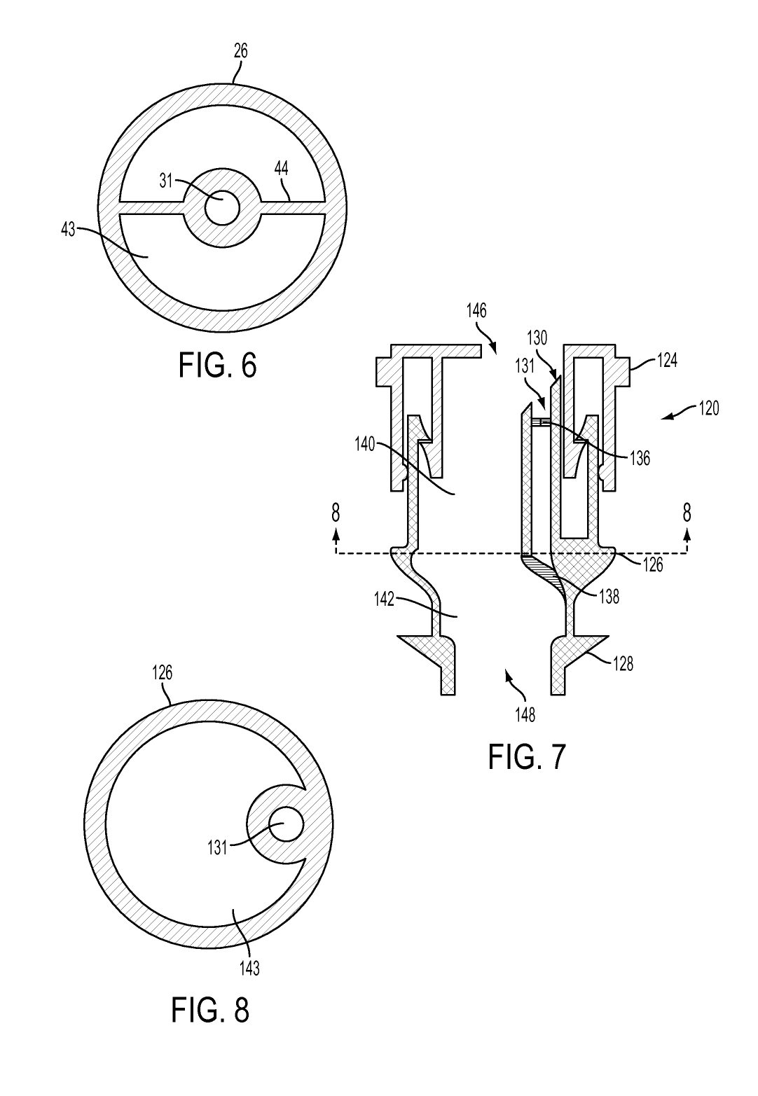

A cross-sectional view of the luer 20 in closed and open positions respectively is shown in FIGS. 4 and 5. The distal housing 26 includes a stem 30 having a stem lumen 31. The stem lumen 31 is fluidly sealed by a pair of elastomeric duckbill valves 36, 38. The valves 36, 38 are biased shut so that they remain closed when subjected to the positive and negative pressure fluctuations of a dialysis procedure, as well as internal body pressure fluctuations. Upon the insertion of a guide wire or a stylet into the stem lumen 31, the valves are configured to open, providing compatibility for wire accessories as needed. In the open position, the proximal opening 46 is in fluid communication with the interior 40 of the proximal housing 24 and the interior 42 of the distal housing 26. A supporting member 44 supports the stem 30, and has openings 43 (as shown in FIG. 6) to allow for fluid communication between the proximal housing 24 interior 40 and the distal housing 26 interior 42. Thus, when in the open configuration, fluid can traverse the interior of the luer housings from the distal opening 48 of the distal housing 26 to the proximal opening 46 of the proximal housing 24. In the closed configuration, fluid cannot traverse to the proximal opening 46 since it is fluidly sealed by the stem 30 protruding through the proximal opening 46. The elastomeric valves 36, 38 can be other types of valves, such as slit valves. A duckbill valve 38 positioned at the distal end of the stem lumen 31 may be especially advantageous since it diverts fluid away from the stem lumen 31 and towards the openings 43. Since fluid communication through the luer can be easily established via slidable actuation by the user, there is no need for extension tubes and clamps, resulting in less proximal end weight, less materials, streamlined manufacturing and a cost savings. Further, patient safety outcomes are improved due to the minimization of extension tube fatigue. In certain embodiments, the luer can be curved up and away from the hub so that when the hub is secured flat to the skin of the patient's body, the luer connection is more easily accessible to the medical provider. Further, the luer and/or hub can be made from a polymer having translucent properties so that the medical provider can properly verify flashback and fluid flow through the proximal ends of the device lumens.

A cross-sectional view of a luer 120 according to an alternative embodiment of the invention is shown in FIG. 7. The luer 120 has a proximal housing 124 and a distal housing 126 that are connected in a slidable mating relationship to each other. The distal housing 126 can have a barb 128 as described in previous embodiments. As shown with additional reference to FIG. 8, the distal housing 126 also includes a stem 130 that is offset from the center of the luer, allowing fluid to flow through a more central opening 143 as it passes through the interior 140 of the luer 120 between the proximal opening 146 and the distal opening 148. The stem 130 can be supported by a bulked-up sidewall to the distal housing 126, further configured to form the stem lumen 131. The offset stem lumen 131 can have a pair of elastomeric valves 136, 138 to fluidly seal the stem lumen 131, while allowing for the traversal of a guide wire or stylet as described in previous embodiments. The top of the stem 130 can also feature angled walls for diverting fluid towards the interior 140 of the luer 120.

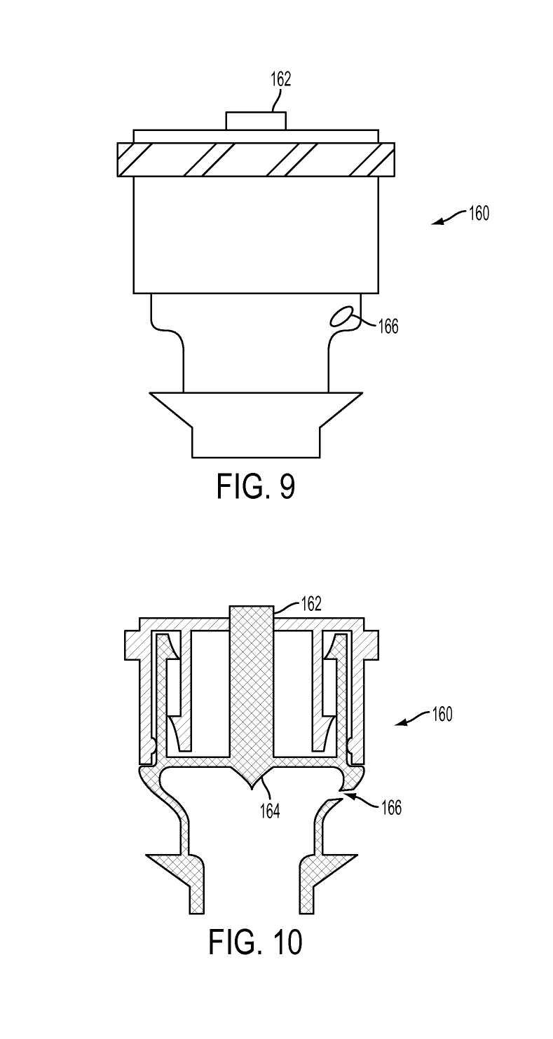

An alternative embodiment of the invention with a modified stem and guide wire access structure is shown in closed configuration in FIGS. 9 and 10. The luer 160 features a stem 162 that does not contain any fluid lumens. The bottom of the stem 164 is geometrically shaped to divert fluid away from the stem, similar to the valve structure shown in the embodiment of FIGS. 4 and 5. A port 166 is provided to allow for the introduction of a guide wire or stylet. The port 166 can be fluidly sealed by a self-sealing elastomeric member, such as a gasket.

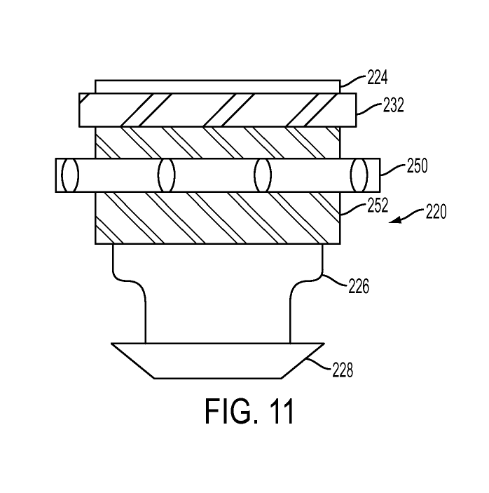

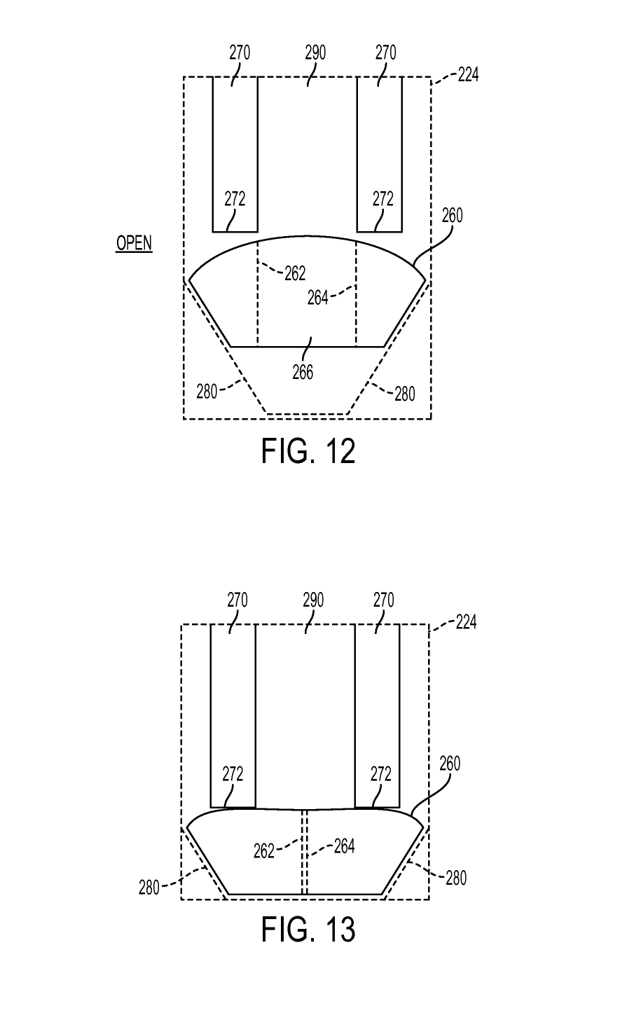

Luers according to certain embodiments of the invention can include a system similar to a tuohy borst valve for closing and opening fluid access through the luer. In the embodiment shown in FIGS. 11-13, a luer 220 includes a proximal housing 224, a distal housing 226 and a barb 228 on the distal housing 226. A tubing threaded element 232 is formed on the luer 220 for attaching external device tubing, such as tubing for a dialysis machine. The luer 220 also includes an actuation threaded element 252 and an actuation collar 250 loaded over the threaded element 252. The actuation collar 250 is configured to raise actuation members 270 in the open position as shown in FIG. 12, and to lower actuation members 270 in the closed position as shown in FIG. 13. The interior of the luer 220 includes an elastomeric insert 260 that has an open lumen 266 in a relaxed configuration. As the actuation collar 250 is twisted towards a closed position, the bottom 272 of the actuation members 270 will push the elastomeric insert 260 along funnel shaped walls 280, closing the lumen walls 262, 264 so that the lumen 266 completely blocks off fluid communication. To reestablish fluid communication, the actuation collar 250 can be twisted towards an open position, allowing the elastomeric insert 260 to expand and move back up the funnel shaped walls 280, allowing the lumen 266 to expand.

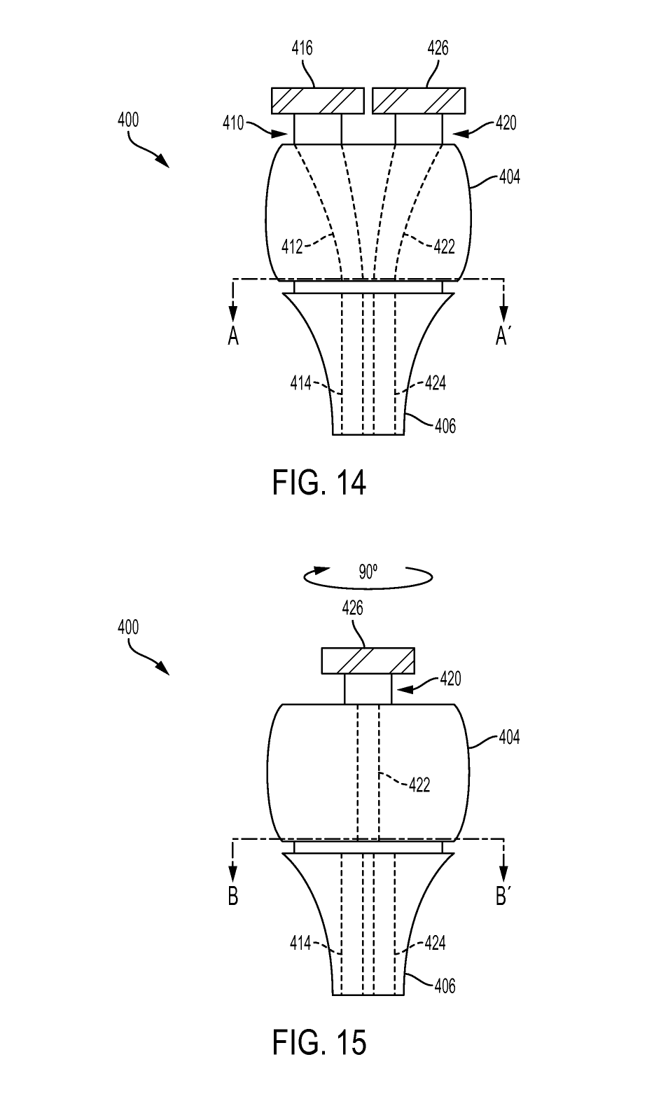

With reference now to FIGS. 14 and 15, an integrated hub and luer configuration is shown that opens and closes fluid access to lumens by applying a twisting or rotational motion to a portion of the hub, according to an embodiment of the invention. The hub 400 includes a first luer 410 and a second luer 420 fixed at the top of the proximal housing component 404 of the hub 400. The luers 410, 420 may each include threaded portions 416, 426 for attachment to external tubing or accessories, such as tubing for a dialysis machine, or a syringe for flushing the device. The hub 400 has a proximal housing component 404 and a distal housing component 406 that are interconnected in a manner such that the proximal housing 404 can be rotated or twisted clockwise and counterclockwise relative to the distal housing 406. The twisting motion allows lumens within the proximal housing 404 and the distal housing 406 to either fully align, partially align or misalign, thereby permitting or prohibiting a fluid to flow between the proximal and distal housing components of the hub. The proximal 404 and distal 406 housings can be joined using known techniques, such as a center-post and snap-fit arrangement.

Specifically, the proximal housing 404 defines a first proximal lumen 412 and a second proximal lumen 422. The first proximal lumen 412 is in fluid communication with the first luer 410, and the second proximal lumen 422 is in fluid communication with the second luer 420. The distal housing 406 also defines a first distal lumen 414 and a second distal lumen 424. As shown in further detail with additional reference to FIGS. 16 and 17, when the hub 400 is in an open position (see FIGS. 14 and 16), the first proximal lumen 412 aligns with the first distal lumen 414, and likewise, the second proximal lumen 422 aligns with the second distal lumen 424, which allows fluid to flow between tubing engaged with the luers 410, 420 and a catheter or other device engaged with one or both distal lumens 414 and 424 at the distal end of the hub 400. The luminal transition between first proximal 412 and distal 414 lumens, as well as the luminal transition between second proximal 422 and distal 424 lumens, can feature common luminal diameters for a smooth transition and continuity in fluid dynamics. Flow of fluid through the hub lumens can be at least partially prohibited by rotating the proximal housing 404 relative to the distal housing 406 (or by rotating the distal housing 406 in a similar fashion), so that the hub 400 approaches a closed position. In the embodiment of FIGS. 16 and 17, an approximately 90° rotation places the hub in a fully closed configuration, whereas less rotation may create limited or partial flow paths, thereby allowing a user to adjust the hub for a number of variable flow rates as desired. In certain embodiments, a locking mechanism, such as notch and groove mating structures, can be used to lock the hub in a particular desired position. In the closed position (see FIGS. 15 and 17), the first proximal lumen 412 is misaligned with the first distal lumen 414, and likewise, the second proximal lumen 422 is misaligned with the second distal lumen 424. In certain embodiments, the mating faces of proximal housing 404 and the distal housing 406 are arranged so that the proximal lumens 412, 422 and the distal lumens 414, 424 interface along a flush surface, forming a fluid-tight interface in the open or closed states. The interface can also utilize other elements such as a compression fitting or elastomeric gaskets to further prevent fluid leakage. Frictional restriction of the twisting motion along the interface to 90 degrees may also serve to ensure that first and second luminal pathways do not cross, and cross-contamination between luminal pathways can be avoided. Although the present embodiment shows a hub 400 configured for two lumens, those having ordinary skill in the art will appreciate that the design can be easily modified for one lumen, or three or more lumens. Likewise, there is no limitation to the size and shape of the hub lumens, and therefore the lumens of the hub may include any straight or curved geometry desired, and any combination of broad or narrow flow regions therethrough.

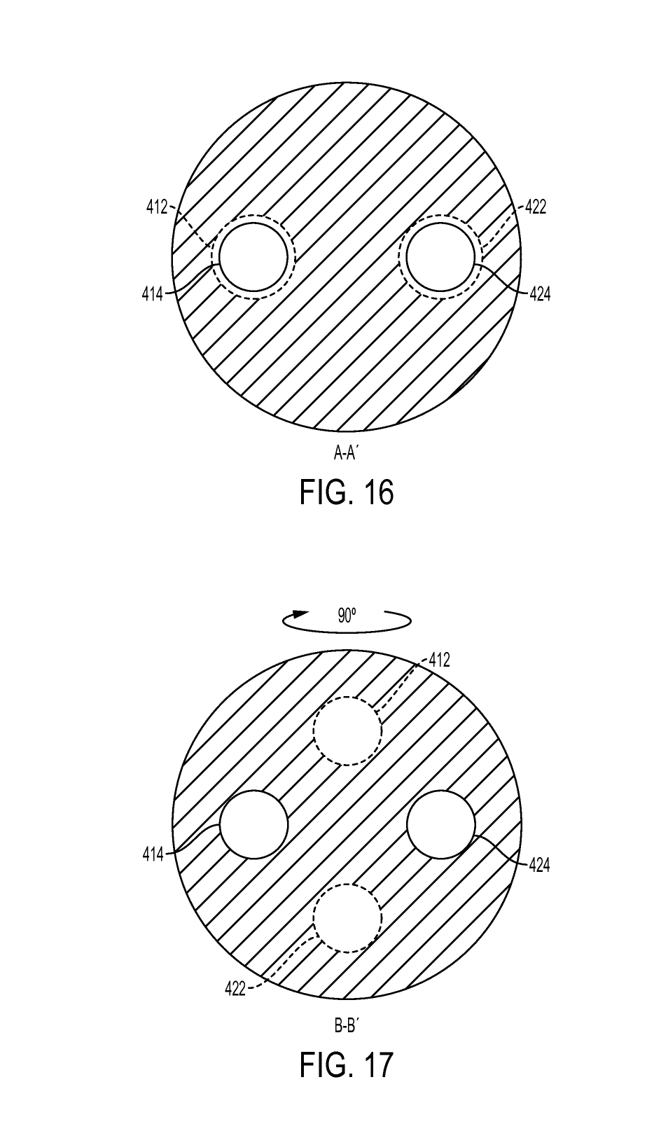

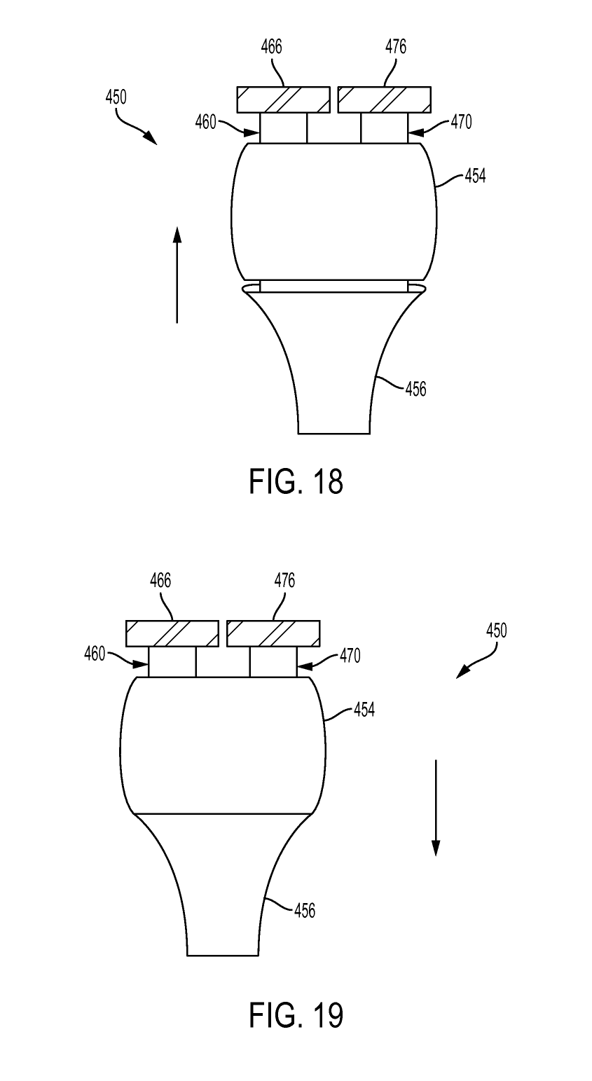

With reference now to FIGS. 18 and 19, an integrated hub and luer configuration is shown that opens and closes fluid access to lumens by applying a push/pull or “snap” motion to the hub, according to an embodiment of the invention. The mechanism for providing fluid access to lumens is in some ways similar to those embodiments described above with reference to FIGS. 2-10, with the exception that the hub housing components are used to actuate the push/pull mechanism, instead of the luers. Advantageously, a multi-lumen catheter utilizing the hub of the current embodiment can open and close all lumens simultaneously by simply actuating the hub, instead of actuating individual luers. Specifically, as shown in FIGS. 18 and 19, the hub 450 has a first luer 460 and a second luer 470 positioned at the top of the hub 450, each with a threaded portion 466, 476 for connection to machine tubing or accessories. The hub has a proximal housing 454 and a distal housing 456 that are configured to allow the proximal housing 454 to pull a short distance away from the distal housing 456, without detaching from the each other. This action may be similar to a push-pull sealing cap used commonly with beverage bottles. An interior lip and ring structure can be used so that a user has to apply a certain force to pull the hub over the lip, ensuring that the hub does not close unintentionally in response to smaller forces.

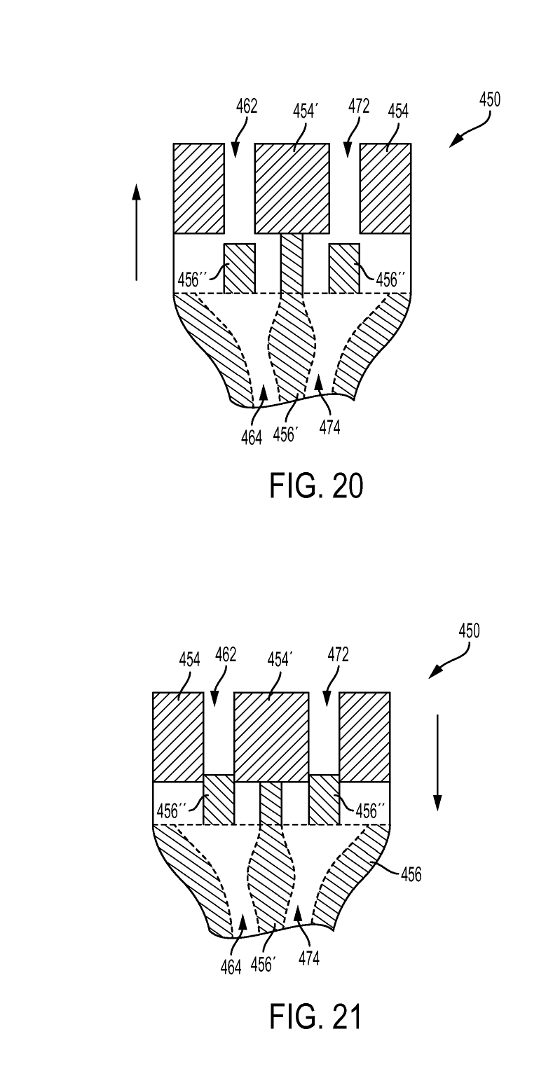

As shown with additional reference to FIGS. 20 and 21, the internal structure of the proximal housing 454 and the distal housing 456 define luminal pathways for transferring fluid between an attached catheter and an auxiliary machine or accessory. The proximal housing 454 defines a first proximal lumen 462 and a second proximal lumen 472. The distal housing 456 defines a first distal lumen 464 and a second distal lumen 474. The first proximal lumen 462 and the second proximal lumen 472 are separated by a central portion or block 454′ of the proximal housing 454. The first distal lumen 464 and the second distal lumen 474 are also separated by a central portion or block 456′ of the distal housing 456. The central portions 454′, 456′ can be used to functionally engage the proximal housing 454 to the distal housing 456 using a slidable mating configuration, such as a center post and center channel arrangement that utilizes a friction fit or detent mechanism, or any other slidable configurations and mechanisms known in the art. When the hub 450 is in an open or “up” position (see FIGS. 18 and 20), fluid communication between the first proximal 462 and distal 464 lumens, and the second proximal 472 and distal 474 lumens is also open, as illustrated in FIG. 20. However, the closed or “down” position (see FIGS. 19 and 21) closes this fluid communication as stem protrusions 456″ from the distal portion 456 plug and fluidly seal the first 462 and second 472 proximal lumens as the proximal housing 454 moves down and mates with the distal housing 456. In certain embodiments, tight fluid seals can be formed by utilizing compression and elastomeric components (e.g. a rigid stem 456″ and corresponding portion of the proximal housing 454 that is elastomeric, or vice versa). In preferred embodiments, the surface contours of the protrusions along the flow paths are contoured, and allow for smooth transitions between adjacent surfaces, facilitating a more laminar flow of fluid, and minimizing turbulent fluid flow. Like the previously described embodiments, the hub design can be easily modified for one lumen, or three or more lumens, and for any size and shape lumen desired.

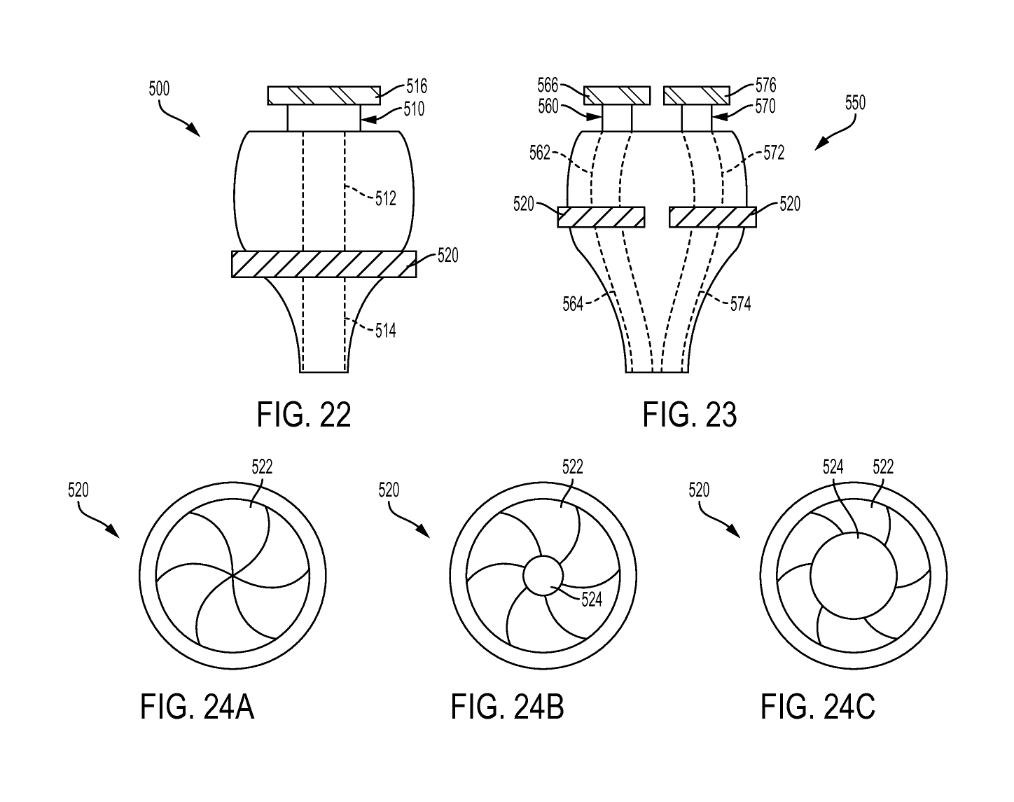

An iris valve can also be used to control fluid flow across luminal pathways in the hub according to an exemplary embodiment of the invention shown in FIGS. 22-24C. With reference first to FIG. 22, a hub 500 for use with a single lumen catheter is shown. The hub 500 has a proximal lumen 512 and a distal lumen 514 separated by an iris valve 520. The top of the hub 500 has a luer 510 and a threaded portion 516 on the luer 510 for connecting to an auxiliary device. An alternative embodiment of the hub features multiple lumens, such as the dual lumen embodiment shown in FIG. 23. The dual lumen hub 550 has a first and second luers 560, 570, each having first and second threaded portions 566, 576 for connecting to an auxiliary device. The first luer 560 is in fluid communication with a first proximal lumen 562 and first distal lumen 564, which are separated by an iris valve 520. The second luer 570 is in fluid communication with a second proximal lumen 572 and a second distal lumen 574, which are also separated by an iris valve 520. The iris valve is adjustable between a closed state, an open state, and partially open states. The valve design itself can be a conventional type iris valve, such as the one described in U.S. Pat. No. 2,321,336 to Tondreau, or similar variations, such as the one described in U.S. Pat. No. 5,158,553 to Berry et al, both herein incorporated by reference. For example, the iris valve 520 may feature a diaphragm 522 that opens and closes in response to rotating a periphery of the valve 520, which is exposed at the surface of the hub 550 (see FIGS. 22 and 23). The iris valve 520 can switch from a closed position (FIG. 24A), which prohibits all flow through the luminal pathway, to a partially open position (FIG. 24B), which allows a restricted flow through the opening 524 and luminal pathway, to a fully open position (FIG. 24C), which allows an unrestricted flow through the opening 524 and luminal pathway. Advantageously, medical professionals can use the iris valves 520 to adjust the flow rate, which may be beneficial for procedures requiring a low or variable flow rates. Further, for multi-lumen catheters, medical professionals can adjust the flow rate of just one or more lumens, so that different lumens on the same catheter have different flow rates (e.g. a high flow rate for CT injection and a low flow rate for introduction of nutrients). This allows high pressure flow rates for a valved catheter, without risking damage to a stationary valve structure (such as an elastomeric slit valve type structure). Additionally, a lock ring may be included that pulls up and locks in, similar to an air control valve. In such embodiments, a “tooth” pattern on the top of the ring may be used, and the bottom of the moving part of the hub may have a mating tooth pattern. Accordingly, when pushed together the mechanism locks. As a more versatile way of customizing flow rates, hospitals could benefit from a cost savings, by stocking less catheters with different dedicated flow rates. In embodiments disclosed herein, any form of symbol, word, number or color identifier can be used on external surfaces of the hubs to indicate on/off or particular flow rate information corresponding to the degree for which a mechanism is partially open.

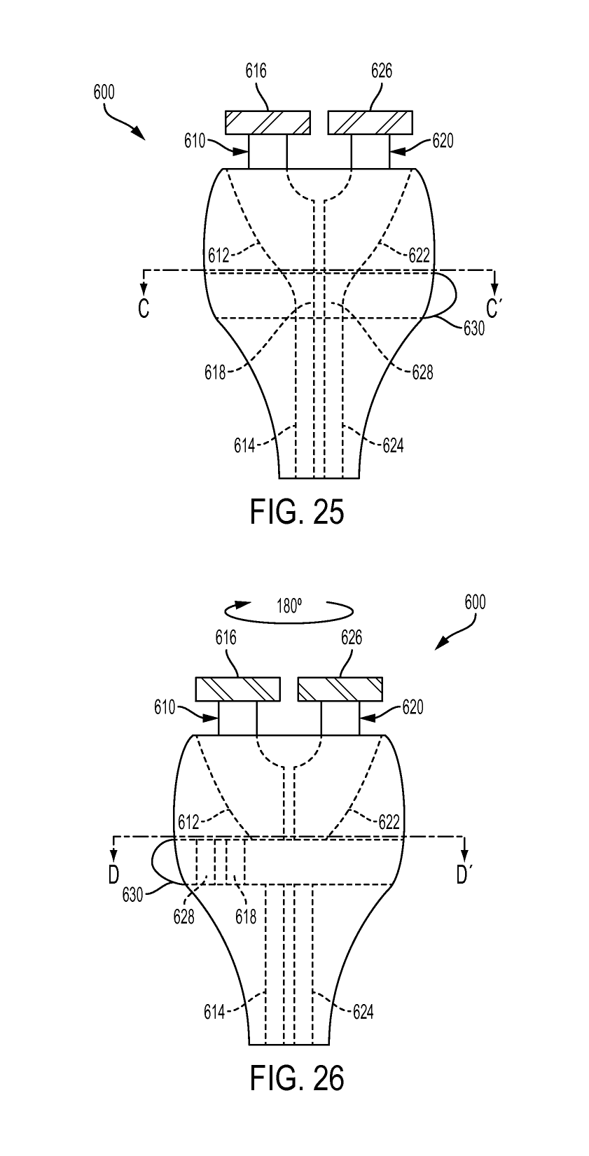

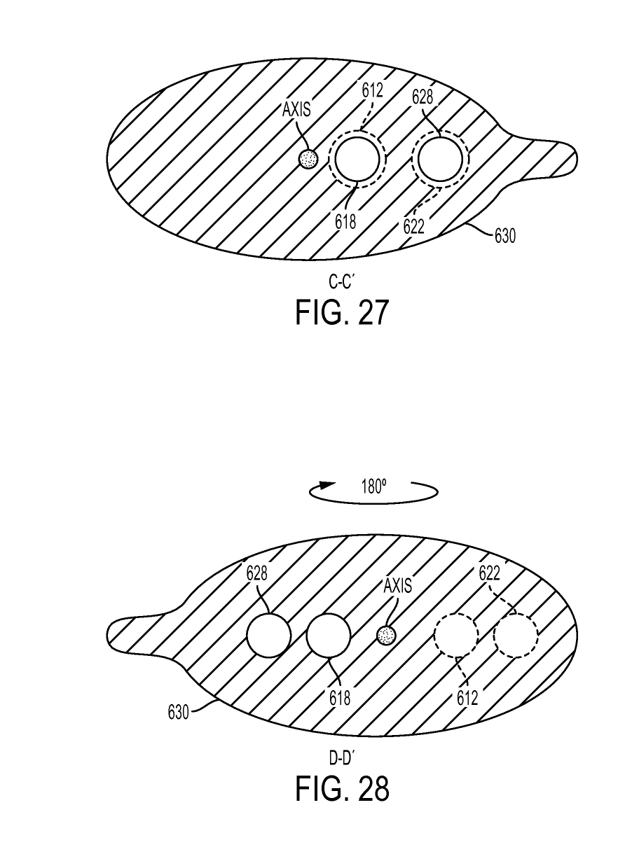

With reference now to FIGS. 25-28, a hub with integrated luers has a switching mechanism for opening and closing fluid access to the lumens, according to an exemplary embodiment of the invention. As shown in FIGS. 25 and 26, the hub 600 has first and second luers 610, 620 at the top, each with their own threaded portions 616, 626. A first proximal lumen 612 is in fluid communication with the first luer 610. To place the hub in an open configuration (FIGS. 25 and 27), a movable switching element 630 connects the first proximal lumen 612 to the first distal lumen 614 via the first switching lumen 618 when the first switching lumen 618 is at least partially aligned with both the first proximal lumen 612 and the first distal lumen 614. Likewise, a second proximal lumen 622 is in fluid communication with the second luer 620. The switching element 630 also connects the second proximal lumen 622 to the second distal lumen 624 via the second switching lumen 628 when the second switching lumen 628 is at least partially aligned with both the second proximal lumen 622 and the second distal lumen 624. Thus, the open position facilitates free flow of fluid through individual luminal pathways. The luminal transition between first proximal lumen 612, the first switching lumen 618 and the first distal lumen 614 can feature common luminal diameters for a smooth transition and continuity in fluid dynamics. The same applies to second proximal lumen 622, the second switching lumen 628 and the second distal lumen 624. To close-off the lumens, the switching element 630 can rotate about its axis, so that the first and second switching lumens 618, 628 are out of alignment with the first and second proximal lumens 612, 622 (see FIGS. 26 and 28), as well as being out of alignment with the first and second distal lumens 614, 622. In this closed position, both the first and second proximal lumens 612, 622 and the first and second distal lumens 614, 622 interface with a solid surface face of the switching element, thereby blocking fluid communication along the luminal pathways. As described in earlier embodiments, the mating surfaces opening to the proximal lumens 612, 622 and the distal lumens 614, 624 interface with the switching element 630 along a flush surface, forming a fluid-tight interface in the open or closed states. The interface can also utilize compression fittings and elastomeric gaskets to discourage fluid leakage. According to the embodiment shown, first and second luminal pathways do not cross, even during switching, and cross-contamination between luminal pathways is avoided. Proximal and distal housings of the hub 600 can mate with the switching element 630 using a variety of well-known mating techniques, such as a snap fit along a central axis element. As will be appreciated by those having ordinary skill in the art, the axis position, shape of the switching lumens, rotational range of motion of the switching element, and position of the switching lumens can vary. In alternative embodiments, switching element 630 may move slidably along a linear path across the hub, or it may move slidably into the interior of the hub. It should be appreciated that the directional movement of switching element 630 is not limited to any particular motion to actuate. The primary requirement is that the switching lumens properly align and misalign to open and close fluid access accordingly. In certain embodiments, the slide may be color coded or have the words “off” and “on” printed on it. For example, when slid into the open position, the word “open” may be visible.

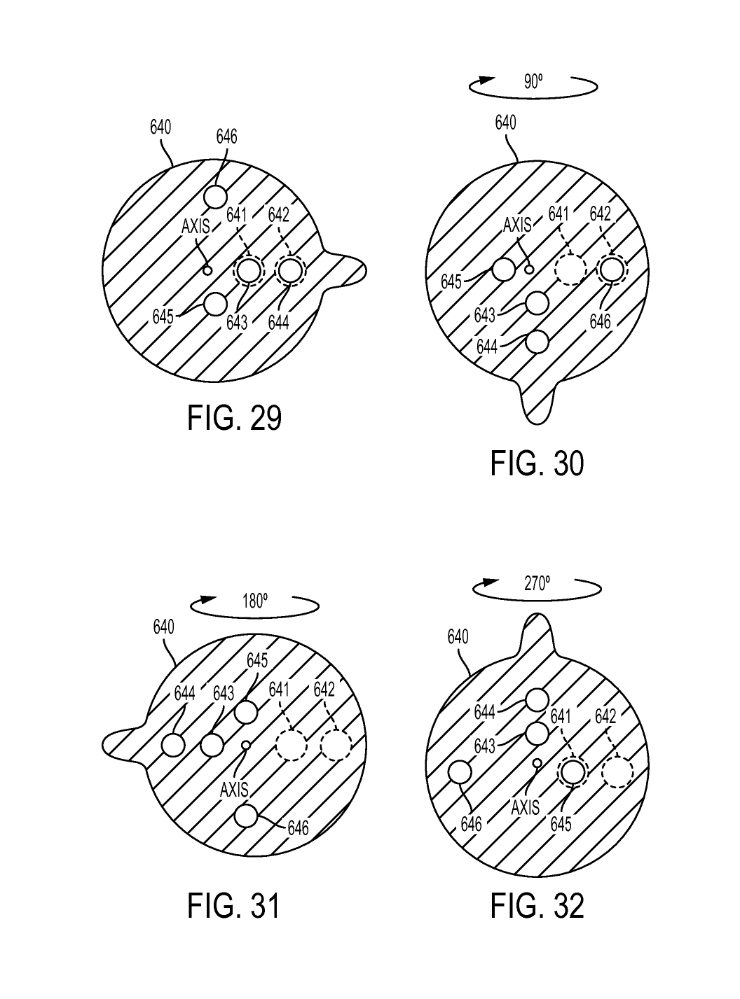

An alternative embodiment for a switching element 640 is shown in FIGS. 29-32. This particular switching element is unique in that it allows for switching between at least four states in a dual lumen catheter, including both lumens open (FIG. 29), both lumens closed (FIG. 31), first lumen open only (FIG. 32), and second lumen open only (FIG. 30). The switching element 640 has a rotational axis in the middle, and in this particular embodiment, is more circular in overall profile. The shape of the switching elements and actuation mechanisms throughout the disclosure can vary, as long as function remains consistent with that described herein. The first and second proximal lumens 641, 642 provide fluid access from a hub to a catheter when they are aligned with a particular switching lumen. Thus, in FIG. 29, both of the first and second proximal lumens 641, 642 are aligned with switching lumens 643, 644, and are therefore both lumens are open for fluid communication to a catheter. In FIG. 30, the switching element 640 is rotated clockwise 90° relative to FIG. 29, and now, only the single second switching lumen 646 is aligned with the second proximal lumen 642, while the first proximal lumen 641 is aligned with a wall of the switching element 640. Thus, the position of the switching element in FIG. 30 will only allow flow through the second proximal lumen 642, while stopping flow through the first proximal lumen 641. In FIG. 31, the switching element 640 is rotated clockwise 180° relative to FIG. 29, and both the first and second proximal lumens 641, 642 are aligned with a wall of the switching element 640, closing fluid flow through both lumens of the catheter. In FIG. 32, the switching element 640 is rotated 270° relative to FIG. 29, and now, the single first switching lumen 645 is aligned with the first proximal lumen 641, while the second proximal lumen 642 is aligned with a wall of the switching element 640. Thus, the position of the switching element in FIG. 32 will only allow flow through the first proximal lumen 641, while stopping flow through the second proximal lumen 642. Symbolic markers such as words, numbers or colors can be printed on the outside of the hub to indicate the various positions. In certain embodiments, the switching element can sustain partially open positions in accordance with the locking and switching techniques described herein.

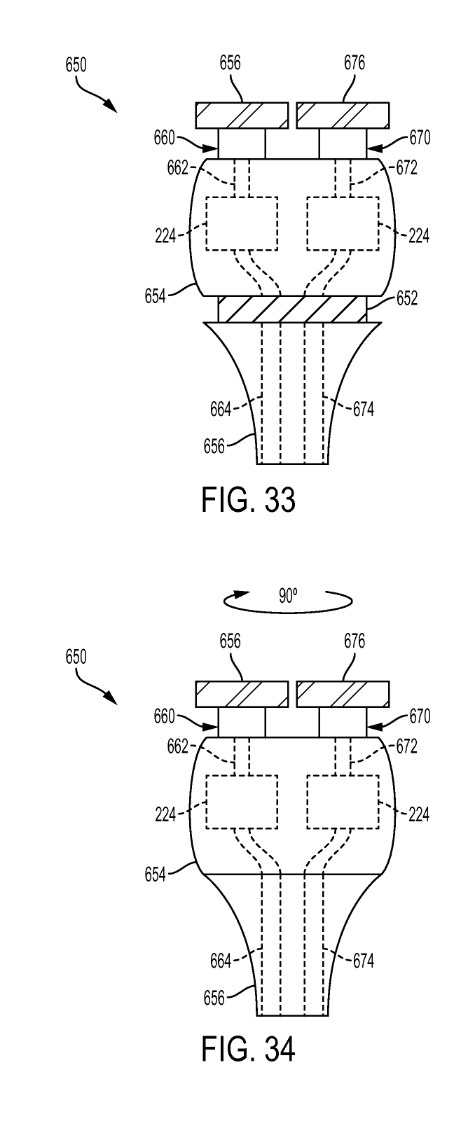

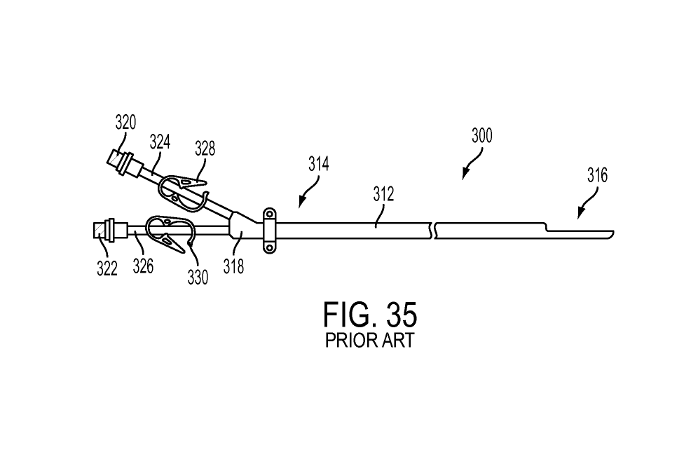

A hub with integrated luer structure that utilizes a rotating hub for actuating multiple tuohy borst valve elements is shown in the exemplary embodiment of FIGS. 33 and 34. The hub 650 has a proximal housing 654 and a distal housing 656. The distal housing 656 includes a threaded element 652, which forms part of the connection with the proximal housing 654. When the proximal housing 654 is rotated, the threaded portion 652 either brings the proximal housing 654 closer or further away from the distal housing 656, depending on the direction of rotation. The proximal housing 654 has first and second luers 660, 670, each including a threaded connection element 656, 676. The first and second luers 660, 670 are connected to first and second proximal lumens 662, 672, respectively. The first and second proximal lumens 662, 672 are in fluid communication with first and second distal lumens 664, 674 via tuohy borst valve elements 224, of which exemplary embodiments were disclosed in a previous embodiment shown in FIGS. 12 and 13. For adaptation to the instant embodiment, actuation members are associated with the proximal housing 654 and the funnel shaped walls are associated with the distal housing 656. It is important to note that the proximal housing 654 spins independently of the first and second proximal lumens 662, 672, and that the movement of the proximal housing 654 is primarily for moving the actuation members 670 up and down. With reference to FIGS. 33 and 34, and in a similar fashion to the valve shown in FIGS. 12 and 13, the interior of the proximal housing 656 includes an elastomeric insert that has an open lumen in a relaxed configuration. As the proximal housing 656 is twisted towards a closed position, the bottom of the actuation members will push the elastomeric insert along funnel shaped walls, closing the lumen walls so that the lumen is fluidly sealed. To reestablish fluid communication, the proximal housing 656 can be twisted towards an open position, allowing the elastomeric insert to expand and move back up the funnel shaped walls, allowing the lumen to once again expand.

Integrated hub and luer configurations disclosed herein that utilize hub actuation for opening and closing lumens can be manufactured by adapting a number of techniques known in the art. The hub can be formed as two or more separate pieces, via an insert molding as an example, or formed as one piece and cut into separate parts based on the type of actuation mechanism. Luers can be molded integrally with the hub, or alternatively, overmolded or attached to the hub in a separate step (utilizing for example a chemical or electrical adhesive method). Although the designs disclosed herein do not require extension tubes, extension tubes can be used to connect the hub and luer if desired. In certain embodiments, hub elements are made from transparent or translucent materials, so that medical professionals can view fluid flow through hub lumens, which may be helpful for monitoring catheter patency and detecting events such as flashback during catheter insertion procedures.

Advantageously, patient care is simplified and improved. Catheters utilizing hubs of the disclosed embodiments can be inserted into a patient with lumens easily opened or closed by simply actuating the hub or luer. A single actuation motion can open or close all lumens. In alternative multi-lumen embodiments, one or more lumens are actuated by a hub actuating mechanism, and one or more lumens are actuated by a luer actuating mechanism. In yet another alternative embodiment, all lumens pass through an actuating hub, and in addition, all lumens have an actuating luer. For example, a triple lumen catheter has an actuating hub for closing all three lumens simultaneously, and all three lumens also have an individual actuating luer mechanism. In this case, medical professionals can easily close (or open) all three lumens at the hub, or, keep the hub open, and selectively close specific lumens using the actuating luer mechanisms. Any procedure traditionally performed via catheter, such as an infusion or aspiration of fluid, can easily be performed utilizing actuating hub and luer devices disclosed herein. Devices are also improved, since eliminating extension tubes and clamps saves manufacturing steps and downsizes the finished device. Devices are also made more reliable, since repeated actuation of mechanisms described herein have a higher durability then repeated clamping on a conventional flexible extension tube.

An exemplary method for use is as follows. The hub or luer mechanism can be opened using a force depending on the type of mechanism described herein (e.g. a pulling for twisting action). In certain embodiments, after a kit or package containing the catheter is opened, a saline syringe is attached to the luer, and the plunger on the syringe is pushed to flush the catheter. The catheter is then ready to advance to the target location using techniques known in the art, such as a Seldinger technique. The syringe is removed after the saline flush. The catheter is advanced to the target location, in some instances, with a block of saline locked within catheter lumens. The actuating mechanism on the hub or luer can be used advantageously to lock the saline, or to fix a guide wire at a particular position. The desired procedure can now commence with the mechanism open (e.g. medication can now be administered, CT can now be injected, and procedures such as dialysis can now be performed). If the catheter remains indwelling, another saline flush can be performed, and the catheter can be closed (with or without a saline lock) using the actuating mechanism. When a subsequent procedure begins at a later time, the actuating mechanism can simply be reopened. This cycle can repeat as long as the catheter is indwelling. Advantageously, repeated opening and closing of the actuation mechanisms described herein to not ware down an extension tube, and extend the life of the catheter. When desired, the catheter can be removed and discarded.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A hub for controlling flow of a fluid along a flow path, comprising:
a proximal housing comprising a first proximal lumen in fluid communication with a first luer; and
a distal housing comprising a first distal lumen;
wherein the proximal housing is attached to the distal housing and configured to rotate relative to the distal housing between an open state and a closed state,
wherein the open state establishes fluid communication between the first proximal lumen and the first distal lumen, and
wherein the closed state blocks fluid communication between the first proximal lumen and the first distal lumen.

2. The hub of claim 1, wherein the proximal housing further comprises a second proximal lumen in fluid communication with a second luer; and
the distal housing further comprises a second distal lumen;
wherein the open state further establishes fluid communication between the second proximal lumen and the second distal lumen, and
wherein the closed state further blocks fluid communication between the second proximal lumen and the second distal lumen.

3. The hub of claim 2, wherein in the open state, the first proximal lumen and the first distal lumen share a first common diameter, and the second proximal lumen and the second distal lumen share a second common diameter.

4. The hub of claim 2, wherein the first luer and the second luer each comprise a threaded element.

5. The hub of claim 2, wherein the first proximal housing is restricted from rotating past a 90 degree range.

6. A dual lumen catheter comprising:
the hub of claim 2; and
a flexible elongate shaft comprising a first catheter lumen and a second catheter lumen;
wherein the elongate shaft is attached to the hub, the first distal lumen is in fluid communication with the first catheter lumen, and the second distal lumen is in fluid communication with the second catheter lumen.

7. A single lumen catheter comprising:
the hub of claim 1; and
a flexible elongate shaft comprising a first catheter lumen;
wherein the elongate shaft is attached to the hub and the first distal lumen is in fluid communication with the first catheter lumen.

8. A vascular access catheter comprising:
an elongate flexible shaft connected to and in fluid communication with a hub, the hub comprising:
a proximal housing comprising a first proximal lumen in fluid communication with a first luer, and
a distal housing comprising a first distal lumen,
wherein the proximal housing is attached to the distal housing and configured to rotate relative to the distal housing between an open state and a closed state,
wherein the open state establishes fluid communication between the first proximal lumen and the first distal lumen, and
wherein the closed state blocks fluid communication between the first proximal lumen and the first distal lumen.

9. The vascular access catheter of claim 8, wherein the proximal housing further comprises a second proximal lumen in fluid communication with a second luer; and
the distal housing further comprises a second distal lumen;
wherein the open state further establishes fluid communication between the second proximal lumen and the second distal lumen, and
wherein the closed state further blocks fluid communication between the second proximal lumen and the second distal lumen.

10. The vascular access catheter of claim 9, wherein in the open state, the first proximal lumen and the first distal lumen share a first common diameter, and the second proximal lumen and the second distal lumen share a second common diameter.

11. The vascular access catheter of claim 9, wherein the first luer and the second luer each comprise a threaded element.

12. The vascular access catheter of claim 9, wherein the first proximal housing is restricted from rotating past a 90 degree range.

13. A hub for controlling flow of a fluid along a flow path, comprising:
a proximal housing comprising a first proximal lumen in fluid communication with a first luer; and
a distal housing comprising a first distal lumen and a first stem;
wherein the proximal housing is attached to the distal housing and configured to slide relative to the distal housing between an open state and a closed state,
wherein the open state establishes fluid communication between the first proximal lumen and the first distal lumen, and
wherein the closed state blocks fluid communication between the first proximal lumen and the first distal lumen by interfacing the first stem with the proximal lumen.

14. The hub of claim 13, wherein the proximal housing further comprises a second proximal lumen in fluid communication with a second luer; and
the distal housing further comprises a second distal lumen and a second stem;
wherein the open state further establishes fluid communication between the second proximal lumen and the second distal lumen, and
wherein the closed state further blocks fluid communication between the second proximal lumen and the second distal lumen by interfacing the second stem with the second proximal lumen.

15. The hub of claim 14, wherein the first proximal lumen and the first stem share a first common axis, and the second proximal lumen and the second stem share a second common axis.

16. The hub of claim 14, wherein the first luer and the second luer each comprise a threaded element.

17. The hub of claim 13, wherein at least one of the first stem and a portion of the proximal housing defining the first proximal lumen comprises an elastomeric material.

18. A vascular access catheter comprising an elongate flexible shaft connected to the hub of claim 13, wherein a first shaft lumen of the elongate flexible shaft is in fluid communication with the first distal lumen.

19. The hub of claim 13, wherein an outer surface of the hub comprises at least one of a word, number, color or symbolic indicator, indicating that the hub is in an open or closed position.

20. The hub of claim 13, wherein the hub includes a transparent or translucent material for viewing blood flow within a hub lumen by viewing through an outer surface of the hub.
